(19) United States
(12) Patent Application Publication
(10) Pub. No.: US 2021/0027865 A1
(43) Pub. Date: Jan. 28, 2021

(54) METHOD AND SYSTEM FOR COMPUTATIONAL MODELLING AND SIMULATION APPLIED TO DRUG CHARACTERIZATION AND/OR OPTIMIZATION

(71) Applicant: INSILICOTRIALS TECHNOLOGIES S.r.l., Italy (IT)

(72) Inventors: Luca EMILI, Trieste (IT); Roberta BURSI, Trieste (IT); Alessia BARETTA, Trieste (IT); Alberto PALAZZIN, Trieste (IT)

(21) Appl. No.: 17/042,771

(22) PCT Filed: Mar. 26, 2019
(86) PCT No.: PCT/IB2019/052450

§ 371 Date: Sep. 28, 2020

(30) Foreign Application Priority Data
Mar. 28, 2018 (IT) 102018000004045

(51) Int. Cl. G16C 20/50; G06F 30/20; G16H 50/50; G16C 20/30; G16B 5/00

(52) U.S. Cl. CPC G16C 20/50; G06F 30/20; G16B 5/00; G16C 20/30; G16H 50/50

(57) ABSTRACT

A method for computational modeling and simulation applied to drug characterization and/or optimization is described.

The method comprises the step of storing on a computational platform 1 digital data for modeling characteristics of an individual D1, digital data for modeling characteristics of an animal D2, digital data for modeling properties of chemical and/or biological compounds and/or drugs D3, digital modeling data of biological targets D4, digital modeling data of disease features and/or therapeutic areas D5 towards which the drug is directed.

The method then comprises the step of receiving selection and/or setting information I which can be entered by a user through a user interface 4 which can be connected to the Internet.

Such information include information on selection and/or definition and/or setting I1 of a pharmacometric and/or physiological model; information on selection and/or definition and/or setting I2 of a chemical and/or pharmacological and/or biological system model; information on selection and/or definition and/or setting I3 of a screening and/or optimization model; information on selection and setting I4 of a modeling and/or simulation type, and/or information on selection and setting I5 of one or more modeling and/or simulation input parameters and information on selection and setting I5 of one or more modeling and/or simulation output parameters I6.

The method then comprises the step of processing, by means of the computational platform 1, the aforementioned types of selection and/or definition and/or setting information to develop a pharmacological and/or physiological model M1 (based on the digital modeling data of an individual D1 and/or digital modeling data of an animal D2), a chemical and/or pharmacological and/or biological and/or therapeutic system model M2 (based on digital modeling data of chemical and/or biological compounds and/or drugs D3 and/or digital modeling data of biological targets D4 and/or digital modeling data of diseases and/or therapeutic areas D5), and a screening and/or optimization model M3 (based on digital modeling data of disease features and/or therapeutic areas D5 and also on the aforementioned digital modeling data of chemical and/or biological compounds and/or drugs D3 and/or digital modeling data of biological targets D4).

Then, the selection and/or setting information I entered by the user is processed for preparing input setting data Din for one or more computational modeling and/or simulation software programs included in the computational platform.

The method then comprises executing computational modeling and/or simulation, by the one or more computational modeling and/or simulation software programs, on the basis of the abovementioned input setting data Din, and based on the pharmacometric and/or physiological model M1, on the chemical and/or pharmacological and/or biological system model M2, and on the screening and/or optimization model M3, to obtain computational modeling and/or simulation output data Dout.

Finally, the method comprises the step of processing the computational modeling and/or simulation output data Dout, on the basis of the information on selection and setting I6 of one or more modeling and/or simulation output parameters, to report the requested modeling and/or simulation results R in a format selected by the user, and the step of providing such requested simulation results through the user interface 4.

### BACKGROUND OF THE INVENTION

### Field of Application

The present invention generally relates to the technical field of computational modeling and simulation by electronic processors/computers in the area commonly defined as “in silico clinical trials” or more simply “in silico trials”.

In particular, the invention refers to a method and system for computational modeling and simulation applied to the research and development of drugs.

More in particular, the invention relates to a method and system for computational modeling and simulation applied to drug characterization and/or optimization.

### Description of the Prior Art

The technical field of “in silico trials” relates to computational modeling and simulation carried out in the pharmacological or medical or biological areas. The use of “in silico trials” increasingly emerges as advantageous to complement, to complete or even to replace experimental tests and evaluations.

In the field of drug characterization and/or optimization, different types of software are known, designed to perform simulations on specific aspects of the identification, definition, testing and verification of the disposition, effectiveness and safety of a drug upon administration.

The terms “drug characterization” and “drug optimization” refer to several aspects and/or phases of the drug research and development process which include, for example, tests of (de-)selection of chemical and/or biological compounds and preclinical and/or clinical trials.

Typically, these phases are successive and distinct from an initial phase of “discovery” (in particular “high-throughput discovery”), which instead falls outside the main applications of the present invention.

In the more specific case of “in silico trials” concerning drug characterization and/or optimization, particularly numerous and complex aspects of biological, pharmacometric, physiological nature, and so on, need consideration. The basic properties of chemical or biological compounds, the mechanism of action, the evaluation of the effects of physiological and organic response in biological targets and/or in preclinical species and/or in humans, the characterization of diseases and/or therapeutic areas, the evaluation of the risk/benefit balance in view of the disease to be treated, the identification of harmful side effects, are just some of the aspects to be studied in depth in the characterization and/or optimization of a drug.

Computational modeling and simulation currently lends help to single specific aspects, among those mentioned above, providing support to experimental activities, which remain necessary and of the utmost importance in the characterization and/or optimization of a drug.

Moreover, known computational simulation software packages or programs, besides being typically focused on very specific objectives, are very complex and expensive. Their use also requires a combination of specialized medical-scientific and IT skills (for example, the operation of controlling numerous computational parameter settings, many of which are related to clinical aspects).

In light of this, in the technical area of “in silico trials” concerning drug characterization and/or optimization, computational modeling and simulation tools capable of treating broad-spectrum and in a more integrated manner drug research and development are needed to support the experimental activity more widely and better, replacing it where possible.

At the same time, these systems and methods of computational modeling and simulation need to be as simple, available and easily accessible as possible to all entities possibly in need thereof (and not only to large entities such as large clinical institutions or large pharmaceutical companies) and adaptable to most of possible cases.

In light of these considerations, a computational platform is urgently needed that allows the creation of a shared and collaborative work environment between different entities which can access the platform, both as data and/or model providers and as a modeling and/or simulation service users, making the methodology effective also in its application-related aspects.

To date, the known solutions of computational simulation do not fully meet all the requisites mentioned above, required for “in silico trials” concerning drug characterization and/or optimization.

### SUMMARY OF THE INVENTION

In light of the above consideration, the object of the present invention is to provide a method for computational modeling and simulation applied to drug characterization and/or optimization, which allows to obviate at least partially the drawbacks mentioned above with reference to the prior art, and meeting the aforementioned requirements particularly felt in the considered technical field.

Such an object is achieved by a method according to claim 1.

Further embodiments of such a method are defined by claims 2-12.

The present invention also relates to a computational modeling and simulation system applied to drug characterization and/or optimization. This system is defined in claim 13.

Further embodiments of the system are defined in claims 14-16.

### BRIEF DESCRIPTION OF THE DRAWINGS

Further features and advantages of such a system and method according to the invention will become apparent from the following description of preferred exemplary embodiments, given by way of a non-limiting example with reference to the accompanying drawing, in which:

FIG. 1 shows a simplified block diagram of a system for the computational modeling and simulation applied to drug characterization and/or optimization, according to an embodiment of the present invention.

### DETAILED DESCRIPTION

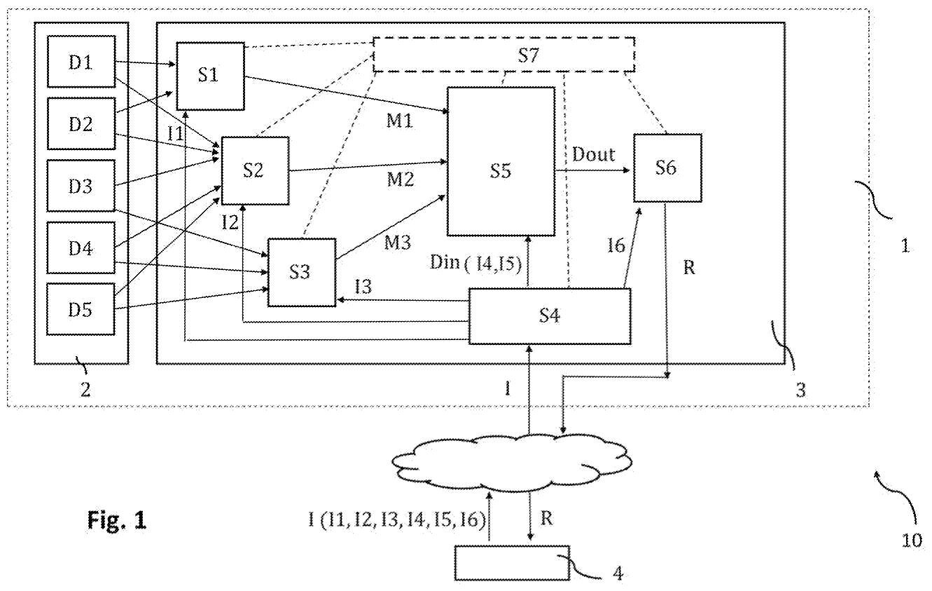

With reference to FIG. 1, a method for computational modeling and simulation is described, applied to the characterization and/or optimization of at least one drug (for example, in individuals and/or animals).

The method, firstly, comprises the steps of storing on a computational platform 1 a first set of digital modeling data D1, a second set of digital modeling data D2, a third set of digital modeling data D3, a fourth set of digital modeling data D4 and a fifth set of digital modeling data D5.

The first set of digital modeling data D1 comprises data representative of chemical, chemical-physical, biological, pharmacological, physiological, genetic, pharmacokinetic, pharmacodynamic and clinical characteristics of a real and/or virtual individual, referred to one or more biological and/or organic and/or functional parts of the individual with which the at least one drug is intended to have interactions and/or referred to one or more effects resulting from such interactions.

For example, the first set D1 of digital modeling data comprises biological and/or pharmacological and/or physiological and/or genetic and/or pharmacokinetic and/or pharmacodynamic and/or clinical data of a real and/or virtual individual, referred to one or more biological and/or organic and/or functional parts of the individual with which the at least one drug is intended to have interactions and/or referred to one or more effects resulting from such interactions.

Such data (also called “modal data”) are obtained, for example, as a result of an interaction between one or more molecules of the drug and the individual.

According to various possible implementation options of the method, the aforementioned biological modal data may comprise, for example, inhibition data of the Interleukin-6 protein involved in the inflammatory or kinetic processes of an antibody acting against a vaccine; the aforementioned pharmacological modal data may include agonistic or inhibitory affinity data of a chemical or biological compound towards a biological target or drug-drug interaction data or dose-response relationships data in specific populations; the aforementioned physiological modal data may include blood pressure or creatinine level or electrocardiogram data following the administration of a drug; the aforementioned genetic modal data may include effect data caused by the genetic polymorphism of the enzyme CYP450 2D6 or by the P-glycoprotein efflux pump or by a specific mononucleotide; the aforementioned pharmacokinetic modal data may include concentration data of a chemical or biological compound in systemic circulation or in tissue or organ; the aforementioned pharmacodynamic and clinical modal data may include HERG2 (Human Epidermal Growth Factor Receptor 2) disease biomarker data or NPS (Neuropathic Pain Scale) score data or ADAS-Cog (Alzheimer's Disease Assessment Scale—Cognitive) score data.

The second set of digital modeling data D2 comprises data representative of chemical, chemical-physical, biological, pharmacological, physiological, genetic, pharmacokinetic, pharmacodynamic characteristics of one or more real and/or virtual animals, referred to one or more biological and/or organic and/or functional parts of the animal with respect to which the at least one drug is intended to be tested and/or referred to one or more effects resulting from testing interactions on animals.

For example, the second set D2 of digital modeling data comprises biological and/or pharmacological and/or physiological and/or genetic and/or pharmacokinetic and/or pharmacodynamic data of one or more real and/or virtual animals, referred to one or more biological and/or organic and/or functional parts of the animal with respect to which the at least one drug is intended to be tested and/or referred to one or more effects resulting from testing interactions on animals.

Such data (also called “modal data”) are obtained, for example, as a result of an interaction between one or more molecules of the drug and the animal.

The above mentioned examples, referred to individuals, also provide examples for biological, pharmacological, physiological, genetic and pharmacokinetic modal data obtained for the characterization and optimization of drugs in animals.

As regards the aforementioned pharmacodynamic modal data in animals, examples may include survival data regarding the progression of breast cancer on transgenic mice of Receptor 2 of the Human Epithelial Growth Factor (HER-2/neu), or efficacy data of a drug against pain assessed in the Flick tail test, or QT prolongation data in the dog due to a ion channel block caused by the administration of a chemical or biological compound.

The third set of digital modeling data D3 comprises data representative of chemical-physical properties and/or function and/or structure and/or behavior of chemical compounds and/or biological compounds and/or drugs to be selected for the research and development of the at least one drug.

The fourth set digital modeling data D4 comprises data representative of chemical-physical properties and/or function and/or structure and/or behavior of biological targets to be selected for the development of the at least one drug.

The fifth set of digital modeling data D5 comprises data representative of chemical, chemical-physical, biological, genetic, physiological, clinical, pharmacological, pharmacodynamic and pharmacokinetic data related to one or more diseases and/or therapeutic areas towards which the drug is directed.

According to different possible implementation options of the method, with reference to the aforementioned data of the fifth set of data D5, the chemical data may include, for example, data of molecular functional groups carrying specific mutagenic or genotoxicproperties; chemical-physical data may include vascular permeability data in a state of acute inflammation or stomach acidity data in gastrointestinal disorders; biological data may include data concerning the interrelationships between the mammary microbiome and malaria or data concerning the role of the Tumor Necrosis Factor α (TNF-α) in the pathogenesis of psoriasis; genetic data may include data regarding specific mutations in the BRCA1 and BRCA2 genes relevant to forms of cancer in organs other than the ovaries and the breast; physiological data may include data regarding the environmental components which lead to obesity; pharmacological data may include data from xenotransplantation studies including liver cancer patients; pharmacodynamic data may include lean muscle data obtained from CT image segmentations in sarcopenic patients; pharmacokinetic data may include data regarding cortisol levels in blood and urine in rheumatoid disease or data regarding the conversion rate of the neurotransmitter Serotonin in depressive disorders.

The method then comprises the step of providing, by means of the computational platform 1, a user interface 4 which can be connected to the Internet, and is configured to allow a user to connect to and interact with the computational platform 1 and with one or more software programs included therein; and the step of receiving selection and/or definition and/or setting information I which can be entered by the user through the user interface 4.

Such selection and/or setting information I comprises information on selection and/or definition and/or setting of a pharmacological and/or physiological model. Such a pharmacometric and/or physiological model comprises a pharmacometric and/or physiological model of an individual based on the aforementioned first stored set D1 of digital data and/or a pharmacometric and/or physiological model of animal based on the aforementioned second stored set D2 of digital data.

The selection and/or setting information also comprises information on selection and/or definition and/or setting I2 of a chemical and/or pharmacological and/or biological and/or therapeutic system model. Such a chemical and/or pharmacological and/or biological and/or therapeutic system may comprise both the pharmacokinetic and/or pharmacodynamic and/or physiological and/or clinical data, and the chemical compounds and/or biological compounds and/or drugs, and the biological targets and/or diseases, previously mentioned, so that the chemical and/or pharmacological and/or biological and/or therapeutic system model is based on the aforementioned first stored set D1 and/or second stored set D2 and/or third stored set D3 and/or fourth stored set D4 and/or fifth stored set D5 of digital modeling data, which can therefore be considered as representative of chemical, chemical-physical, pharmacological, biological, genetic, pharmacometric, physiological, clinical characteristics of the chemical and/or pharmacological and/or biological and/or therapeutic system.

The selection and/or setting information further comprise information on selection and/or definition and/or setting I3 of a screening and/or optimization model based on the aforementioned third stored set D3 and/or fourth stored set D4 and/or fifth stored set D5 of digital modeling data; and also information on selection and setting I4 of a type of simulation, and/or information on selection and setting I5 of one or more input simulation parameters, and information I6 on one or more output simulation parameters.

The method then comprises the step of processing, by means of the computational platform 1, the information on selection and/or definition and/or setting I1 of a pharmacometric and/or physiological model to develop a pharmacometric and/or physiological model M1 based on the aforesaid first stored set D1 and/or second stored set D2 of digital modeling data.

According to several possible implementation methods of the method, the pharmacometric model M1 may consist of a model which describes the distribution and clearance of a drug whose pattern is target-mediated, or may consist of a model which identifies the relationship between the levels of an analgesic drug in systemic circulation and their effect on reducing chronic pain in a population of cancer patients.

According to several possible implementation options of the method, the physiological model M1 may consist of a model which describes the concentration of a drug in systemic circulation as a function of the expression levels of metabolizing enzymes and organ development and with which the dosage of such a drug to be administered to a pediatric patient with specific characteristics of age, weight and gender may be calculated, or in a model which describes the pharmacological effect of the blockage of the P-glycoprotein transporter on the distribution and pattern of a particular drug.

The method then comprises the step of processing, by means of the computational platform 1, the information on selection and/or definition and/or setting I3 of a chemical and/or pharmacological and/or biological and/or therapeutic system model to develop a chemical and/or pharmacological and/or biological system model M2 based on the aforementioned first stored set D1 and/or second stored set D2 and/or third stored set D3 and/or on fourth stored set D4 and/or on fifth stored set D5 of digital modeling data.

According to different possible implementation options of the method, the aforementioned model M2 of chemical and/or pharmacological and/or biological and/or therapeutic system may refer to a chemical system which assigns the absolute configuration of a chiral chemical compound based on the analysis of molecular Vibrational Circular Dichroism spectra; and/or may refer to a pharmacological system which describes the distribution of a drug between blood and/or plasma, cerebrospinal fluid and central nervous system; and/or may refer to a biological system which represents the immune system (B cells and plasma cells, cytotoxic T cells, dendritic cells, macrophages, antigens, antibodies, cytokines) and possible interactions with tumor cells and/or vaccines; and/or may refer to a therapeutic system concerning the nutritional status of a patient that integrates data on lean muscle mass, medical history, results of quality of life questionnaires and a nutritional recommendation.

The method then includes the step of processing, by means of the computational platform, the information on selection and/or definition and/or setting I3 of a screening and/or optimization model to develop a screening and/or optimization model M3, based on the aforementioned fifth stored set D5 of digital modeling data and based on the aforementioned third stored set D3 and/or fourth stored set D4 of digital data.

According to different possible implementation options of the method, the aforementioned screening and/or optimization model M3 may be a model adapted to identify patients with the genetic profile of interest for a specific clinical study; or a model adapted to identify possible relationships between adverse and/or toxic side effects of chemical and/or biological compounds of interest and the biological targets forming part of their mechanism of action.

The screening and/or optimization model M3 may include search algorithms and/or artificial intelligence algorithms adapted to identify and connect the function and/or structure and/or behavior of chemical compounds and/or biological compounds and/or drugs and/or biological targets and/or diseases which are relevant to the development of the at least one drug.

The method then comprises the step of processing, by means of the computational platform 1, the selection and/or setting information I entered by the user to define input setting data Din (depending on I4, I5) for one or more computational modeling and simulation software programs included in the computational platform.

The method then comprises the step of executing computational modeling and simulation, by the one or more computational modeling and/or simulation software programs, on the basis of the abovementioned input setting data Din, and of the abovementioned pharmacometric and/or physiological model M1, chemical and/or pharmacological and/or biological and/or therapeutic system model M2, and screening and/or optimization model M3, to obtain output data Dout of the computational simulation.

Finally, the method comprises the step of processing the output data Dout of the computational simulation, on the basis of the information on the selection and setting of one or more output simulation parameters I6, to express the requested simulation results R in a format selected by the user and the step of providing such requested simulation results by means of the user interface 4.

The simulation results R include information apt to identify and/or connect function and/or structure and/or behavior of chemical compounds and/or biological compounds and/or drugs and/or biological targets and/or diseases which are relevant to the development of the at least one drug.

The aforementioned “digital models” M1, M2, M3 may include, for example, structured digital data sets apt to digitally represent the respective entities indicated above; these structured digital data sets are apt to be treated or processed by computational simulation software programs or packages.

The method can operate on software programs or packages of computational simulation known per se, for example NONMEM®, Monolix, PK-SIM and MATLAB®.

According to an embodiment of the method, the step of providing a user interface comprises providing a plurality of user-selectable templates associated with respective types of simulation. Each of these templates comprises: a plurality of input parameters which can be selected for the simulation, in which each parameter is associated with a respective range of permitted values that are appropriate for the feasibility of the simulation, within which a parameter value can be set; a plurality of selectable output parameters, comprising the quantities requested as the output result; a plurality of displaying and reporting options, which can be selected by the user to choose the format of the results.

The aforementioned input and output parameters depend on the type of simulation required and performed.

In this embodiment, the templates can be predefined in the most diverse ways, with regard to the choice of the simulation input and output parameters and the relative permitted intervals, and regarding the displaying and reporting of the results, allowing much easier management of the simulation by the user.

According to an embodiment of the method, the selection and/or setting information I which can be entered by the user also comprises parameters aiming to define and elaborate (i.e., for defining and/or elaborating) the pharmacometric and/or physiological model M1, and/or parameters aiming to define and elaborate (i.e., for defining and/or elaborating) the chemical and/or pharmacological and/or biological and/or therapeutic system model M2, and/or parameters aiming to define and customize (i.e., for defining and/or customizing) algorithms of the optimization model using artificial intelligence M3; and also comprising parameters aiming to select and/or define (i.e., for selecting and defining) real or virtual patients, or populations of real or virtual patient; and/or parameters aiming to set up (i.e., for setting up) computational aspects of the simulation.

According to various possible implementation options, the selection and/or setting information I which can be entered by the user comprises initial conditions and boundary conditions for the simulation, and/or parameters relating to the numerical and/or analytical method used by the modeling and/or computational simulation software.

According to an embodiment, the method further comprises the step of obtaining digital modeling data of a pharmacometric and/or physiological model and/or digital modeling data of a chemical and/or pharmacological and/or biological and/or therapeutic system and/or digital modeling data of screening or optimization model by selecting from a plurality of digital pharmacological and/or physiological models and/or digital models of chemical and/or pharmacological and/or biological and/or therapeutic and/or screening and/or optimization models stored in a digital library 2 of the computational platform 1 and/or pre-loaded by the user on the computational platform; and, furthermore, the method comprises the step of obtaining digital modeling data D1 comprising biological, pharmacological, physiological, genetic, pharmacokinetic, pharmacodynamic and clinical data of a real and/or virtual individual by selecting from a plurality of digital models of real or virtual patients stored in the digital library 2 of the computational platform 1 and/or pre-loaded by the user on the computational platform.

In this embodiment, the method is applied to a large plurality of models which can be stored or pre-loaded in the digital library, which therefore can be known per se.

In a particular embodiment of the method, the digital modeling data of a real individual are anonymized and/or de-identified and/or pseudonymized.

According to several possible implementation options of the method, the computational modeling and simulation comprises modeling and simulations that searches for biological targets and/or that searches for chemical compounds and/or that searches for biological compounds and/or that searches for drugs.

The method can be applied in conjunction with a plurality of computational simulations (belonging to the aforementioned categories), characterized by the most various complexity, procedures, number of iterations, number and type of computational modeling and simulation programs, and so on. Below, some significant examples of computational modeling and simulations included in the method will be explicitly indicated.

According to an implementation example of the method, the modeling and computational simulations comprise modeling and simulations for the characterization and/or optimization of the aforesaid at least one drug, and/or simulations for the analysis and prediction of the behavior of the at least one drug on a population of real or virtual patients, and/or simulations for a personalized evaluation of the effects of the at least one drug on a specific real or virtual patient, and/or simulations for evaluations of the safety and/or efficacy and/or compliance with current on safety and/or efficacy regulations.

According to an application example of the method, the computational modeling and/or simulations further comprise simulations for the analysis and prediction of the behavior of one or more drugs on a population of real or virtual animals, and/or for a customized evaluation of the effects of one or more drugs on a specific real or virtual animal.

According to another application example of the method, the modeling and computational simulations comprise modeling and simulations for the design and/or development of the chemical-physical and/or pharmacological and/or biological properties of one or more chemical compounds and/or for evaluations of the safety and/or efficacy and/or compliance with current safety and/or efficacy regulations.

According to a further application example of the method, the computational modeling and simulations comprise modeling and simulations for the analysis and/or identification and/or characterization of biological targets for research and/or safety and/or efficacy evaluations.

According to another example of application of the method, the modeling and computational simulations comprise search algorithms which operate on digital modeling data.

According to implementation examples of the method, the software simulation programs or packages used to carry out some of the aforementioned simulations may comprise suites of processing solutions based on artificial intelligence, known per se, provided by the main suppliers of software solutions of this type (for example, Microsoft, Google and Amazon).

With reference again to FIG. 1, a system 10 for computational modeling and simulation applied to the characterization and/or optimization of at least one drug, comprising a computational platform 1, is described.

This computational platform comprises at least one digital library 2 and one or more electronic processing components 3 in which one or more software programs or applications (S1-S7) are stored and can be executed.

The digital library 2 stores a first set of digital modeling data D1, a second set of digital modeling data D2, a third set of digital modeling data D3, a fourth set of digital modeling data D4 and a fifth set of digital modeling data D5.

The first set D1 of digital modeling data comprises biological, pharmacological, genetic, physiological, pharmacokinetic, pharmacodynamic and clinical data (also referred to as “modal data”) of a real and/or virtual individual, referred to one or more biological and/or organic and/or functional parts of the individual with which the at least one drug is intended to have interactions and/or referred to one or more effects resulting from said interactions.

The second set D2 of digital modeling data comprises biological, pharmacological, genetic, physiological, pharmacokinetic, pharmacodynamic data (also referred to as “modal data”) of one or more real and/or virtual animals, referred to one or more biological and/or organic and/or functional parts of the animal with respect to which the at least one drug is intended to be tested and/or referred to one or more effects resulting from testing interactions on an animal.

The third set of digital modeling data D3 comprises data representative of chemical-physical properties and/or function and/or structure and/or behavior of chemical compounds and/or biological compounds and/or drugs to be selected for the research and development of the at least one drug.

The fourth set digital modeling data D4 comprises data representative of chemical-physical properties and/or function and/or structure and/or behavior of biological targets to be selected for the development of the drug.

The fifth set of digital modeling data D5 comprises data representative of chemical, chemical-physical, biological, physiological, clinical, pharmacological, genetic, pharmacodynamic and pharmacokinetic data related to one or more diseases and/or therapeutic areas towards which the drug is directed.

The aforesaid one or more electronic processing components 3 are configured to perform, by means of the one or more software programs or applications (S1-S7) stored on and executed therein, the actions of: providing a user interface 4 which can be connected to the Internet and is configured to allow a user to connect to and interact with the digital data processing platform 1 and with one or more software programs included therein; then, receiving selection and/or setting information I which can be entered by the user through the user interface 4. Such selection and/or setting information includes information on the selection and/or definition and/or setting I1 of a pharmacometric and/or physiological model; information on the selection and/or definition and/or setting I2 of a chemical and/or pharmacological and/or biological and/or therapeutic system model; information on the selection and/or definition and/or setting I3 of a screening and/or optimization model; information on the selection and setting I4 of a simulation type, and/or information on the selection and setting I5 of one or more simulation input parameters and information on the selection and setting I6 of one or more simulation output parameters. Further details on such selection and/or setting information have been illustrated above, in the context of the description of the method according to the invention.

The one or more electronic processing components 3 are further configured to carry out, by means of the one or more software programs or applications (S1-S7), the following further processing aimed at preparing a plurality of digital models: processing the information on the selection and/or definition and/or setting I1 of a pharmacometric and/or physiological model to develop a pharmacometric and/or physiological model M1 based on the aforementioned first stored set D1 and/or second stored set D2 of digital modeling data; moreover, processing the information on the selection and/or definition and/or setting I2 of a chemical and/or pharmacological and/or biological and/or therapeutic system model to develop a chemical and/or pharmacological and/or biological and/or therapeutic system model M2 based on the aforementioned first stored set D1 and/or second stored set D2 and/or third stored set D3 and/or fourth stored set D4 and/or fifth stored set D5 of digital modeling data; in addition, processing the information on the selection and/or definition and/or setting I3 of a screening and/or optimization model to develop a screening and/or optimization model M3, based on the fifth stored set D5 of digital modeling data and based on the third stored set D3 and/or fourth stored set D4 of digital data; such a screening and/or optimization model M3 may comprise search algorithms and/or artificial intelligence algorithms apt to identify and to connect the function and/or structure and/or behavior of chemical compounds and/or biological compounds and/or drugs and/or biological targets and/or diseases that are relevant to the development of the at least one drug.

The aforementioned one or more electronic processing components 3 are further configured to carry out, by means of the one or more software programs or applications S1-S7, the further actions of: processing the aforementioned selection and/or setting information I entered by the user for preparing input setting data Din (dependent on I4, I5) for one or more computational simulation and modeling software programs S5 included in the computational platform 1; then, carrying out the modeling and/or computational simulation, by the one or more computational simulation software programs S5, on the basis of the input setting data Din, on the basis of the pharmacometric and/or physiological model M1, on the basis of the chemical and/or pharmacological and/or biological and/or therapeutic system model M2 and on the basis of the screening and/or optimization model M3, to obtain output data of the modeling and/or computational simulation Dout; finally, processing the output data of the modeling and/or computational simulation Dout, based on the information on selection and setting I6 of one or more modeling and/or simulation output parameters, to report the requested simulation results R in a format selected by the user; and providing the requested modeling and/or simulation results by means of the user interface 4. The simulation results R include information suitable to identify and/or to characterize and/or to connect function and/or structure and/or behavior of chemical compounds and/or biological compounds and/or drugs and/or biological targets and/or diseases that are relevant to the development of the drug.

According to an embodiment of the system 10, the aforementioned one or more software programs or applications (S1-S7) of the computational platform comprise: one or more user interface management software programs S4; one or more computational modeling and/or simulation software programs S5, configured to perform modeling and computational simulation when executed by a computer; one or more processing software programs (S1, S2, S3, S4, S6), configured to perform the steps of processing the information on selection and/or definition and/or setting of a pharmacological and/or physiological model, processing the information on selection and/or definition and/or setting of a chemical and/or pharmacological and/or biological and/or therapeutic system model, processing the information on selection and/or definition and/or setting of a screening and/or optimization model, processing the selection and/or setting information entered by the user to prepare input setting data Din (dependent on I4, I5) for the computational modeling and/or simulation software programs and processing the output data of the computational modeling and/or simulation Dout to report the requested simulation results R in a format selected by the user.

According to an implementation option, the computational platform 1 further comprises a software program S7 of the PIDO (Process Integration and Design Optimization) type, configured to manage the flow process of the software programs comprised in the computational platform and optimize the computational modeling and/or simulations.

With reference to the aforementioned software programs included in the system, the most various options, per se known, referred to the implementation, partition, storage, execution of such software programs, as individual programs or as packages of programs and/or software modules interacting with each other, may be contemplated.

Several options are possible for the practical implementation of the system, from an infrastructural point of view. Among these, concentrated or distributed platforms, based on one or more interacting servers and/or computers, may be contemplated.

For example, the system can be implemented using a distributed cloud computing platform.

According to further implementation examples, the system is configured to execute a method according to any one of the embodiments of the method described above.

According to an implementation example, one or more software programs S1, S4 and S5 may be configured to carry out the steps of processing the information on definition and setting of a pharmacometric model in a particular animal species.

As apparent from the description, the object of the present invention is fully achieved by the method and system described above, by virtue of their functional and structural features.

In fact, by virtue of the features described above, the computational modeling and simulation method of the present invention can support testing/trial activities, in the field of drug research and development, in a more effective and integrated manner, and to increase and extend the role of computational modeling and simulation in this field (which in turn can provide advantages in terms of time and cost reduction and performance improvement in the pharmacological development).

Moreover, due to the processing carried out by the computational platform and the user interface that are provided (as previously described in detail), this computational simulation method can be used in a simple manner, and is easily accessible to anyone (appropriately authorized) who can access the platform. The user interface provides the broadest possibilities for the modeling and/or simulation personalization and adaptation, and at the same time provides guidance and facilitates the set-up of the modeling and/or simulation.

The features mentioned above, in turn, allow to create a shared and collaborative work environment between different entities which can access the platform, playing the role of digital data and/or model providers and/or requesting a modeling and/or simulation service, thus making the methodology effective also in its application aspects.

Similar advantages can be identified with reference to the system, described above, capable of executing the method of the invention.

In order to meet incidental needs, those skilled in the art may make several changes, adjustments and adaptations to the embodiments of the system and method according to the invention, and may replace elements with others which are functionally equivalent, without departing from the scope of the following claims. Each of the features described as belonging to a possible embodiment can be achieved irrespective of the other embodiments described.

1-16. (canceled)

17. A method for computational modeling and simulation applied to characterization and optimization of at least one drug, comprising the steps of:
storing on a computational platform a first set of digital modeling data comprising biological, pharmacological, genetic, physiological, pharmacokinetic, pharmacodynamic and clinical data of a real and/or virtual individual, referred to one or more biological and/or organic and/or functional parts of the individual with which the at least one drug is intended to have interactions and/or referred to one or more effects resulting from said interactions;
storing on the computational platform a second set of digital modeling data comprising biological, pharmacological, genetic, physiological, pharmacokinetic, pharmacodynamic data of one or more real and/or virtual animals, referred to one or more biological and/or organic and/or functional parts of the animal with respect to which the at least one drug is intended to be tested and/or referred to one or more effects resulting from testing interactions;
storing on the computational platform a third set of digital modeling data representative of chemical-physical properties and/or function and/or structure and/or behavior of chemical Preliminary Amendment Submitted with Application compounds and/or biological compounds and/or drugs to be selected for the research and development of the at least one drug;
storing on the computational platform a fourth set of digital modeling data representative of chemical-physical properties and/or function and/or structure and/or behavior of biological targets to be selected for the research and development of said at least one drug;
storing on the computational platform a fifth set of digital modeling data representative of chemical, chemical-physical, biological, physiological, genetic, clinical, pharmacological, pharmacodynamic and pharmacokinetic data related to one or more diseases and/or therapeutic areas towards which said at least one drug is directed;
providing, by means of the computational platform, a user interface which can be connected to the Internet and configured to allow a user to connect to and interact with the computational platform and with one or more software programs included therein;
receiving selection and/or definition and/or setting information which can be entered by the user by means of the user interface, wherein the selection and/or setting information comprises:
information on selection and/or definition and/or setting of a pharmacometric and/or physiological model, comprising a pharmacometric and/or physiological model of an individual based on said first stored set of digital modeling data and/or on a pharmacometric and/or physiological model of animal based on said second stored set of digital modeling data;
information on selection and/or definition and/or setting of a chemical and/or pharmacological and/or biological system model based on said first stored set and/or on said second stored set and/or on said third stored set and/or on said fourth stored set and/or on said fifth stored set of digital modeling data, representative of chemical, chemical-physical, pharmacological, biological, genetic, physiological properties of said chemical and/or pharmacological and/or biological system;
information on selection and/or definition and/or setting of a screening and/or optimization model;
information on selection and setting of a type of simulation, and/or information on selection and setting of one or more input simulation parameters, and information on one or more output simulation parameters;
processing, by means of the computational platform, said information on selection and/or definition and/or setting of a pharmacometric and/or physiological model to develop a pharmacometric and/or physiological model based on said first stored set of digital modeling data and/or based on said second stored set of digital modeling data;
processing, by means of the computational platform, said information on selection and/or definition and/or setting of a chemical and/or pharmacological and/or biological system model to develop a chemical and/or pharmacological and/or biological system model based on said first stored set and/or based on said second stored set and/or based on said third stored set and/or based on said fourth stored set and/or based on said fifth stored set of digital modeling data;
processing, by means of the computational platform, said information on selection and/or definition and/or setting of a screening and/or optimization model to develop a screening and/or optimization model, based on said fifth stored set of digital modeling data and/or based on said third stored set and/or on said fourth stored set of digital data; wherein the screening and/or optimization model comprises search algorithms and/or artificial intelligence algorithms adapted to identify and connect the function and/or structure and/or behavior of chemical compounds and/or biological compounds and/or drugs and/or biological targets and/or diseases which are relevant to the development of the at least one drug;
processing, by means of the computational platform, said selection and/or setting information entered by the user to define input setting data for one or more computational simulation software programs included in the computational platform;
executing computational simulation, by the one or more computational simulation software programs, on the basis of said input setting data, of said pharmacometric and/or physiological model, of said chemical and/or pharmacological and/or biological system model and of said screening and/or optimization model, to obtain output data of the computational modeling and/or simulation;
processing the output data of the computational modeling and/or simulation, on the basis of said information on the selection and setting of one or more output modeling and/or simulation parameters, to report the requested modeling and/or simulation results in a format selected by the user; wherein the simulation results comprise information apt to identify and/or to characterize and/or to connect function and/or structure and/or behavior of chemical compounds and/or biological compounds and/or drugs and/or biological targets and/or diseases which are relevant to the development of the at least one drug;
providing the requested modeling and/or simulation results by means of the user interface.

18. A method according to claim 17, wherein the step of providing a user interface comprises providing of a plurality of user-selectable templates, associated with respective types of simulation, and wherein each template comprises:
a plurality of input parameters which can be selected for the simulation, each parameter being associated with a respective range of permitted values that are appropriate for the feasibility of the simulation, within which a parameter value can be set;
a plurality of selectable output parameters, comprising the quantities requested as an output result;
a plurality of displaying and reporting options, which can be selected by the user to choose the format of the results.

19. A method according to claim 17, wherein the selection and/or setting information (I) which can be entered by the user further comprises:
parameters aiming to define and/or aiming to elaborate the pharmacometric and/or physiological model,
and/or parameters aiming to define and/or aiming to elaborate the chemical and/or pharmacological and/or biological system model,
and/or parameters aiming to define and/or aiming to customize algorithms of the computational model using artificial intelligence;
and/or parameters aiming to select and/or aiming to define real or virtual patients, or populations of real or virtual patients;
and/or parameters aiming to set up computational aspects of the simulation.

20. A method according to claim 19, wherein the selection and/or setting information (I) which can be entered by the user comprises initial conditions and boundary conditions to perform computational modeling and simulation,
and/or parameters related to the numerical method used by the computational simulation software.

21. A method according to claim 17, further comprising the steps of:
obtaining digital modeling data of a pharmacometric and/or physiological model and/or digital modeling data of a chemical and/or pharmacological and/or biological system and/or digital modeling data of a screening or optimization model, by selecting from a plurality of digital pharmacometric and/or physiological models and/or digital models of chemical and/or pharmacological and/or biological systems and/or screening or optimization models stored on a digital library of the computational platform and/or pre-loaded by the user on the computational platform;
obtaining digital modeling data comprising biological, pharmacological, genetic, physiological, pharmacokinetic, pharmacodynamic and clinical data of a real and/or virtual individual by selecting from a plurality of digital models of real or virtual patients stored in the digital library of the computational platform and/or pre-loaded by the user on the computational platform.

22. A method according to claim 17, wherein the digital modeling data of a real individual are anonymized and/or de-identified and/or pseudonymized.

23. A method according to claim 17, wherein the computational modeling and/or simulation comprises modeling and/or simulation that searches for biological targets and/or that searches for chemical compounds and/or that searches for biological compounds and/or that searches for drugs.

24. A method according to claim 17, wherein the computational modeling and/or simulations comprise modeling and/or simulation for the characterization and/or optimization of said at least one drug,
and/or modeling and/or simulation for the analysis and prediction of the behavior of at least one drug on a population of real or virtual patients,
and/or modeling and/or simulation for a personalized evaluation of the effects of the at least one drug on a specific real or virtual patient,
and/or modeling and/or simulation for evaluations of the safety and/or efficacy and/or compliance with current safety and/or efficacy regulations.

25. A method according to claim 24, wherein the computational modeling and/or simulation further comprise modeling and/or simulation for the analysis and prediction of the behavior of one or more drugs on a population of real or virtual animals, and/or for a customized evaluation of the effects of one or more drugs on a specific real or virtual animal.

26. A method according to any claim 17, wherein the computational modeling and/or simulation comprise modeling and/or simulation for the design and/or development of one or more chemical compounds and/or for the analysis and prediction of the chemical-physical and/or pharmacological and/or biological properties of one or more chemical compounds and/or for the evaluations of the safety and/or efficacy and/or compliance with current safety and/or efficacy regulations.

27. A method according to claim 17, wherein the computational modeling and/or simulation comprise modeling and/or simulation for the analysis and/or identification and/or characterization of biological targets for research and/or safety and/or efficacy evaluations.

28. A method according to claim 17, wherein the computational modeling and/or simulation comprise search algorithms operating on the digital modeling data.

29. A system for computational modeling and simulation applied to the characterization and optimization of at least one drug, comprising a computational platform, wherein the computational platform comprises:
a digital library on which the following is stored:
a first set of digital modeling data comprising biological, pharmacological, genetic, physiological, pharmacokinetic, pharmacodynamic and clinical data of a real and/or virtual individual, referred to one or more biological and/or organic and/or functional parts of the individual with which the at least one drug is intended to have interactions and/or referred to one or more effects resulting from said interactions;
a second set of digital modeling data comprising biological, pharmacological, genetic, physiological, pharmacokinetic, pharmacodynamic data of one or more real and/or virtual animals, referred to one or more biological and/or organic and/or functional parts of the animal with respect to which the at least one drug is intended to be tested and/or referred to one or more effects resulting from testing interactions;
a third set of digital modeling data representative of chemical-physical properties and/or function and/or structure and/or behavior of chemical compounds and/or biological compounds and/or drugs to be selected for the research and development of the at least one drug;
a fourth set of digital modeling data representative of chemical-physical properties and/or function and/or structure and/or behavior of biological targets to be selected for the development of said at least one drug;
a fifth set of digital modeling data representative of chemical, chemical-physical, biological, physiological, clinical, pharmacological, genetic, pharmacodynamic and pharmacokinetic data related to one or more diseases and/or therapeutic areas towards which said at least one drug is directed;
one or more electronic processing components configured to perform, by means of one or more software programs or applications stored on and executed therein, the actions of:
providing a user interface which can be connected to the Internet and is configured to allow a user to connect to and interact with the computational platform of digital data and with one or more software programs included therein;
receiving selection and/or setting information which can be entered by the user by means of the user interface, wherein the selection and/or setting information comprises:
information on selection and/or definition and/or setting of a pharmacometric and/or physiological model, comprising a pharmacometric and/or physiological model of an individual based on said first stored set of digital modeling data and/or comprising a pharmacometric and/or physiological model of animal based on said second stored set of digital modeling data;
information on selection and/or definition and/or setting of a chemical and/or pharmacological and/or biological system model based on said first stored set and/or based on said second stored set and/or based on said third stored set and/or based on said fourth stored set and/or based on said fifth stored set of digital modeling data, representative of chemical, chemical-physical, pharmacological, biological, genetic, physiological properties of said chemical and/or pharmacological and/or biological system;
information on selection and/or definition and/or setting of a screening and/or optimization model;
information on selection and setting of a modeling and/or simulation type, and/or information on the selection and setting of one or more input modeling and/or simulation parameters, and information on the selection and setting of one or more output modeling and/or simulation parameters;
processing said information on selection and/or definition and/or setting of a pharmacometric and/or physiological model to develop a pharmacometric and/or physiological model based on said first stored set of digital modeling data and/or based on said second stored set of digital modeling data;
processing said information on selection and/or definition and/or setting of a chemical and/or pharmacological and/or biological system model to develop a chemical and/or pharmacological and/or biological system model based on said first stored set and/or based on said second stored set and/or based on said third stored set and/or based on said fourth stored set and/or based on said fifth stored set of digital modeling data;
processing said information on selection and/or definition and/or setting of a screening and/or optimization model to develop a screening and/or optimization model , based on said fifth stored set of digital modeling data and/or based on said third stored set of digital modeling data and/or based on said fourth stored set of digital modeling data; wherein the screening and/or optimization model comprises search algorithms and/or artificial intelligence algorithms apt to identify and to connect the function and/or structure and/or behavior of chemical compounds and/or biological compounds and/or drugs and/or biological targets and/or diseases that are relevant to the development of the at least one drug;
processing said selection and/or setting information entered by the user for preparing input setting data for one or more computational modeling and/or simulation software programs included in the computational platform;
executing computational modeling and simulation, by the one or more computational modeling and/or simulation software programs, on the basis of said input setting data, on the basis of said pharmacometric and/or physiological model, on the basis of said chemical and/or pharmacological and/or biological system model, and on the basis of said screening and/or optimization model, to obtain output data of the computational modeling and/or simulation;
processing the output data of the computational modeling and/or simulation, on the basis of said information on the selection and setting of one or more output modeling and/or simulation parameters, to report the requested simulation results in a format selected by the user; wherein the simulation results comprise information apt to identify and/or to characterize and/or to connect function and/or structure and/or behavior of chemical compounds and/or biological compounds and/or drugs and/or biological targets and/or diseases which are relevant to the development of the at least one drug;
providing the requested modeling and/or simulation results by means of the user interface.

30. A system according to claim 29, wherein said one or more software programs or applications of the computational platform comprise:
one or more user interface management software programs;
one or more computational modeling and/or simulation software programs, configured to perform the computational modeling and/or simulation when executed by a computer;
one or more software processing programs, configured to perform said steps of processing the information on selection and/or definition and/or setting of a pharmacometric and/or physiological model, processing the information on selection and/or definition and/or setting of a chemical and/or pharmacological and/or biological model , processing the information on selection and/or definition and/or setting of a screening and/or optimization model, processing the selection and/or setting information entered by the user for preparing input setting data for the computational modeling and/or simulation software programs and processing the output data of the computational modeling and/or simulation to report the requested modeling and/or simulation results in a format selected by the user.

31. A system according to claim 30, wherein the computational platform further comprises a software program of the PIDO (Process Integration and Design Optimization) type, configured to manage the flow process of the software programs included in the computational platform and optimize the computational simulations.

32. A system according to claim 29, implemented by means of a distributed cloud computing platform.
